(19) United States
(12) Patent Application Publication
(10) Pub. No.: US 2019/0382842 A1
(43) Pub. Date: Dec. 19, 2019

(54) DETECTION AND TREATMENT OF DILATED CARDIOMYOPATHY IN THE DOBERMAN PINSCHER

(71) Applicant: NORTH CAROLINA STATE UNIVERSITY, RALEIGH, NC (US)

(72) Inventor: Kathryn M. Meurs, Raleigh, NC (US)

(21) Appl. No.: 16/439,877

(22) Filed: Jun. 13, 2019

Related U.S. Application Data
(60) Provisional application No. 62/684,286, filed on Jun. 13, 2018.

(51) Int. Cl. C12Q 1/6883; G01N 33/68

(52) U.S. Cl. CPC C12Q 1/6883; G01N 33/6893; C12Q 2600/124; G01N 2800/325; C12Q 2600/136; C12Q 2600/156; A01K 2227/10

(57) ABSTRACT

The invention relates to methods for identifying a Doberman pinscher dog as having familial dilated cardiomyopathy (DCM) or having an increased risk of developing familial DCM, comprising detecting in nucleic acid from the Doberman pinscher dog a missense mutation in the titin gene (TTN) (DCM2 mutation). The invention further provides methods for treating DCM and methods for delaying the onset or progression of DCM and/or delaying the development of symptoms of DCM in a Doberman pinscher dog.

### STATEMENT OF PRIORITY

This application claims the benefit, under 35 U.S.C. § 119(e), of U.S. Provisional Application Ser. No. 62/684,286, filed Jun. 13, 2018, the entire contents of which are incorporated by reference herein.

### STATEMENT REGARDING ELECTRONIC FILING OF A SEQUENCE LISTING

A Sequence Listing in ASCII text format, submitted under 37 C.F.R. § 1.821, entitled 5051-938_ST25.txt, 468 bytes in size, generated on Jun. 10, 2019 and filed via EFS-Web, is provided in lieu of a paper copy. This Sequence Listing is hereby incorporated herein by reference into the specification for its disclosures.

### FIELD OF THE INVENTION

The invention relates to methods for identifying and treating Doberman dogs having dilated cardiomyopathy (DCM).

### BACKGROUND OF THE INVENTION

Dilated cardiomyopathy (DCM) is a primary heart muscle disease characterized by left ventricular dilation and systolic dysfunction. It has been identified as one of the most common forms of familial cardiomyopathy in human beings, characterized by both genetic and allelic heterogeneity.¹ Mutations have been identified in over 60 genes, particularly those that encode sarcomeric and cytoskeletal proteins. Mutations in the titin gene (TTN) are reported most commonly.²⁻⁶

The dog is a natural animal model of familial DCM, used to study different aspects of the disease from its pathophysiology to stem cell therapy.⁷⁻⁸ The Doberman pinscher is one of the breeds of dogs most commonly affected by familial DCM.⁹ Dilated cardiomyopathy in the Doberman pinscher is characterized by left ventricular dilation and systolic dysfunction, with subsequent development of both atrial and ventricular tachyarrhythmias.^{10,11} Familial aspects of this disease are well described.¹²⁻¹⁴

The present invention overcomes previous shortcomings in the art by providing methods of determining the risk of developing DCM in the Doberman pinscher dog and the opportunity for early intervention to inhibit and/or delay the development of the disease and/or prolong the life of the dog.

### SUMMARY OF THE INVENTION

One aspect of the invention provides a method of identifying a Doberman pinscher dog as having familial dilated cardiomyopathy (DCM) or having an increased risk of developing familial DCM, comprising detecting in nucleic acid from the Doberman pinscher dog a missense mutation in the titin gene (TTN) (DCM2 mutation), thereby identifying the Doberman pinscher dog as having DCM or having an increased risk of developing DCM, wherein the missense mutation is a C→T mutation on canine chromosome 36:22,321,955 (e!Ensembl Gene Accession No. ENSCAFT00000022319) (CanFAM 3.1).

A second aspect of the invention provides a method of identifying a Doberman pinscher dog as having familial dilated cardiomyopathy (DCM) or as having an increased risk of developing familial DCM, comprising detecting in nucleic acid of the Doberman pinscher dog (a) a missense mutation in the titin gene (TTN) (DCM2 mutation), and (b) a splice site mutation in pyruvate dehydrogenase kinase 4 (PDK4) (DCM1 mutation), wherein the missense mutation is a C-+T mutation on canine chromosome 36:22,321,955 (ENSCAFT0000022319) (CanFAM 3.1) and the splice site mutation in PDK4 is a 16 base pair deletion in the 5′ donor splice site of intron 10 of PDK4 on canine chromosome 14:20829667-20829682(e!Ensembl Gene Accession No. ENSCAFG00000002129 (=PDK4)) (CanFAM3.1).

A third aspect of the invention provides a method of identifying and treating a Doberman pinscher dog at increased risk for death due to familial dilated cardiomyopathy (DCM), comprising: a) detecting in nucleic acid from the Doberman pinscher dog a missense mutation in the titin gene (TTN) (DCM2 mutation), thereby identifying the Doberman pinscher dog as having an increased risk for death due to DCM; and b) treating the Doberman pinscher dog having an increased risk for death due to DCM prior to symptom and/or disease development to inhibit and/or delay symptom and/or disease development (e.g., slow progression of the disease) and prolong the life of the Doberman pinscher dog.

A fourth aspect of the invention provides a method of identifying and treating a Doberman pinscher dog at increased risk for death due to familial dilated cardiomyopathy (DCM), comprising: a) detecting in nucleic acid of the Doberman pinscher dog (i) a missense mutation in the titin gene (TTN) (DCM2 mutation), and (ii) a splice site mutation in pyruvate dehydrogenase kinase 4 (PDK4) (DCM1 mutation), thereby identifying the Doberman pinscher dog as having an increased risk for death due to DCM; and b) treating the Doberman pinscher dog having an increased risk for death due to DCM prior to symptom and/or disease development to inhibit and/or delay symptom and/or disease development (e.g., slow progression of the disease) and prolong the life of the Doberman pinscher dog.

A fifth aspect of the invention provides a method of treating a Doberman pinscher dog having familial dilated cardiomyopathy (DCM) or having an increased risk of developing familial DCM, comprising (a) administering to the dog a therapeutically effective amount of an inodilator, an angiotensin converting enzyme (ACE) inhibitor and/or a ventricular antiarrhythmic agent; (b) supplementing the diet of the dog with free fatty acids; (c) monitoring the dog for symptoms (observation) and/or for occult disease; and/or (d) directing/advising (other guidance) the dog's caretaker on exercise considerations/exercise monitoring, wherein the Doberman pinscher dog has a missense mutation in the titin gene (TTN) (DCM2 mutation).

A sixth aspect of the invention provides a method of treating a Doberman pinscher dog having familial dilated cardiomyopathy (DCM) or having an increased risk of developing familial DCM, comprising (a) administering to the dog a therapeutically effective amount of an inodilator, an angiotensin converting enzyme (ACE) inhibitor and/or a ventricular antiarrhythmic agent; (b) supplementing the diet of the dog with free fatty acids; (c) monitoring the dog for symptoms (observation) and/or for occult disease; and/or (d) directing/advising (other guidance) the dog's caretaker on exercise considerations/exercise monitoring, wherein the Doberman pinscher dog has (i) a missense mutation in the titin gene (TTN) (DCM2 mutation) and (ii) a splice site mutation in pyruvate dehydrogenase kinase 4 (PDK4) (DCM1 mutation).

A seventh aspect of the invention provides a method of delaying the onset and/or progression and/or delaying the development of symptoms of familial dilated cardiomyopathy (DCM) in a Doberman pinscher dog that is pre-symptomatic for familial DCM and having a missense mutation in the titin gene (TTN) (DCM2), comprising treating the Doberman pinscher dog by (a) administering to the dog a therapeutically effective amount of an inodilator, an angiotensin converting enzyme (ACE) inhibitor and/or a ventricular antiarrhythmic agent; (b) supplementing the diet of the dog with free fatty acids; (c) monitoring the dog for symptoms (observation) and/or for occult disease; and/or (d) directing/advising (other guidance) the dog's caretaker on exercise considerations/exercise monitoring, thereby delaying the onset and/or progression of DCM and/or delaying the development of symptoms of DCM as compared to a Doberman pinscher dog that has the mutation in the titin gene (DCM2) but which has not been and/or is not being treated for DCM as described herein.

A eighth aspect of the invention provides a method of delaying the onset and/or progression and/or delaying the development of symptoms of familial dilated cardiomyopathy (DCM) in a Doberman pinscher dog that is pre-symptomatic for familial DCM and having (a) a missense mutation in the titin gene (TTN) (DCM2 mutation) and (b) a splice site mutation in pyruvate dehydrogenase kinase 4 (PDK4 (DCM1 mutation), comprising treating the Doberman pinscher dog by comprising treating the Doberman pinscher dog by (a) administering to the dog a therapeutically effective amount of an inodilator, an angiotensin converting enzyme (ACE) inhibitor and/or a ventricular antiarrhythmic agent; (b) supplementing the diet of the dog with free fatty acids; (c) monitoring the dog for symptoms (observation) and/or for occult disease; and/or (d) directing/advising (other guidance) the dog's caretaker on exercise considerations/exercise monitoring, thereby delaying the onset and/or progression and/or delaying the development of symptoms of DCM as compared to a Doberman pinscher dog that has the mutation in the titin gene (DCM2) and the mutation in the pyruvate dehydrogenase kinase 4 (PDK4) gene (DCM1) but which has not been and/or is not being treated for DCM as described herein.

A ninth aspect of the invention provides a method of selectively breeding Doberman pinscher dogs to decrease the frequency of familial dilated cardiomyopathy (DCM) in a Doberman pinscher dog breeding population, comprising: (a) identifying Doberman pinscher dogs in the breeding population that do not have a missense mutation in the titin gene (TTN) (DCM2); and (b) selectively breeding the Doberman pinscher dogs identified in (a), thereby decreasing the frequency of familial dilated cardiomyopathy (DCM) in the Doberman pinscher dog population.

A tenth aspect of the invention provides a method of selectively breeding Doberman pinscher dogs to decrease the frequency of familial dilated cardiomyopathy (DCM) in a Doberman pinscher dog breeding population, comprising: (a) identifying Doberman pinscher dogs in the breeding population that do not have a missense mutation in the titin gene (TTN) (DCM2) and do not have a splice site mutation in pyruvate dehydrogenase kinase 4 (PDK4) (DCM1); and (b) selectively breeding the Doberman pinscher dogs identified in (a), thereby decreasing the frequency of familial dilated cardiomyopathy (DCM) in the Doberman pinscher dog population thereby decreasing the frequency of familial dilated cardiomyopathy (DCM) in the Doberman pinscher dog population.

These and other aspects of the invention are set forth in more detail in the description of the invention below.

### BRIEF DESCRIPTION OF THE DRAWINGS

FIG. 1. Panel A) M mode echocardiogram of the left ventricle of an unaffected Doberman pinscher (top) and an affected Doberman pinscher (bottom). Note reduced interventricular septum (IVS) and left ventricular free wall (LVFW) motion and increased left ventricular lumen (LFFW) size in affected dog. Panel B) Chromatogram of DNA sequence from unaffected dog (top) and affected dog (bottom). Arrows indicate location of DNA variant (C in unaffected dog, T in affected dog).

FIG. 2. Pedigree from a family of Doberman pinschers with dilated cardiomyopathy. Circles indicate females, squares indicate males. White symbols indicate unaffected dogs, black symbols indicate affected dogs, patterned symbols indicate unknown phenotype. Symbols with a line indicate deceased animals. The dog's genotype is indicated next to each symbol. TT, homozygous variant; CT, heterozygous variant; CC, wildtype homozygous.

FIG. 3. Representative electron micrographs of cardiac tissue from two Doberman pinchers control dogs (CC) and two DCM dogs homozygous for the TTN mutation (TT). In sections from control dogs (left 2 panels obtained from 2 different animals) the sarcomeres show a regular structure, while in the TT sections (middle 2 panels and right 2 panels obtained from 2 different animals) sarcomere misalignment is observed, as well as disarrangement of myofilaments, wide spaces between myofibrils, Z-disk streaming with increased Z-disk thickness (arrows), and variation in 1-band width. (Scale bar=1 μm)

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “increased,” “enhance,” “enhanced,” “enhancing,” and “enhancement” (and grammatical variations thereof) describe an elevation of at least about 25%, 50%, 75%, 100%, 150%, 200%, 300%, 400%, 500% or more as compared to a control.

As used herein, the terms “reduce,” “diminish,” “decrease” and “inhibit” (and grammatical variations thereof), describe, for example, a decrease of at least about 5%, 10%, 15%, 20%, 25%, 35%, 50%, 75%, 80%, 85%, 90%, 95%, 97%, 98%, 99%, or 100% as compared to a control.

A “therapeutically effective” amount as used herein is an amount that provides some improvement or benefit to the subject Doberman pinscher dog. Alternatively stated, a “therapeutically effective” amount is an amount that will provide some alleviation, mitigation, or decrease in at least one symptom in the subject (e.g., in the case of DCM, a reduction in or a delay in at least one symptom may include, but is not limited to, coughing, difficulty breathing/shortness of breath, episodes of collapse; a reduction or delay in developing occult disease may include, but is not limited to, ventricular arrhythmia, supraventricular arrhythmia, decrease in heart function, heart enlargement, identification of a heart murmur, and/or elevation in brain natriuretic peptide or troponin levels). Those skilled in the art will appreciate that the therapeutic effects need not be complete or curative, as long as some benefit is provided to the subject Doberman pinscher dog.

By the terms “treat,” “treating,” or “treatment of,” it is intended that the severity of the subject's condition is reduced or at least partially improved or modified and that some alleviation, mitigation, inhibition or decrease in at least one symptom is achieved and/or alleviation, mitigation, inhibition or decrease in development of disease is achieved.

As used herein, “nucleic acid,” “nucleotide sequence,” and “polynucleotide” are used interchangeably and encompass both RNA and DNA, including cDNA, genomic DNA, mRNA, synthetic (e.g., chemically synthesized) DNA or RNA and chimeras of RNA and DNA. The term polynucleotide, nucleotide sequence, or nucleic acid refers to a chain of nucleotides without regard to length of the chain. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases.

An “isolated polynucleotide” is a nucleotide sequence that is not immediately contiguous with nucleotide sequences with which it is immediately contiguous (one on the 5′ end and one on the 3′ end) in the naturally occurring genome of the organism from which it is derived. Thus, in one embodiment, an isolated nucleic acid includes some or all of the 5′ non-coding (e.g., promoter) sequences that are immediately contiguous to a coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant DNA that is part of a hybrid nucleic acid encoding an additional polypeptide or peptide sequence.

The term “oligonuclcotide” refers to a nucleic acid sequence of at least about five nucleotides to about 500 nucleotides (e.g. 5, 6, 7, 8, 9, 10, 12, 15, 18, 20, 21, 22, 25, 30, 35, 40, 45, 50, 55, 60, 65, 70, 75, 80, 85, 90, 100, 125, 150, 175, 200, 250, 300, 350, 400, 450 or 500 nucleotides). In some embodiments, for example, an oligonucleotide can be from about 15 nucleotides to about 30 nucleotides, or about 20 nucleotides to about 25 nucleotides, which can be used, for example, as a primer in a polymerase chain reaction (PCR) amplification assay and/or as a probe in a hybridization assay or in a microarray. Oligonucleotides of this invention can be natural or synthetic, e.g., DNA, RNA, PNA, LNA, modified backbones, etc., as are well known in the art.

The term “isolated” can refer to a nucleic acid or polypeptide that is substantially free of cellular material, viral material, and/or culture medium (when produced by recombinant DNA techniques), or chemical precursors or other chemicals (when chemically synthesized). Moreover, an “isolated fragment” is a fragment of a nucleic acid or polypeptide that is not naturally occurring as a fragment and would not be found in the natural state. “Isolated” does not mean that the preparation is technically pure (homogeneous), but it is sufficiently pure to provide the polypeptide or nucleic acid in a form in which it can be used for the intended purpose.

An “allele” as used herein refers to one of two or more alternative forms of a nucleotide sequence at a given position (locus) on a chromosome. An allele can be a nucleotide present in a nucleotide sequence that makes up the coding sequence of a gene and/or an allele can be a nucleotide in a non-coding region of a gene (e.g., in a genomic sequence). A subject's genotype for a given gene is the set of alleles the subject happens to possess. As noted herein, an individual can be heterozygous or homozygous for any allele of this invention.

The terms “increased risk” and “decreased risk” as used herein define the level of risk that a subject has of developing DCM, as compared to a control subject that does not have the mutation in the titin gene (DCM2) or does not have the mutation in the titin gene (DCM2) and the mutation in the pyruvate dehydrogenase kinase 4 (PDK4) gene (DCM1) in the control subject's nucleic acid.

A sample of this invention can be any sample containing nucleic acid of a subject, as would be well known to one of ordinary skill in the art. Nonlimiting examples of a sample of this invention include a cell, a body fluid, a tissue, biopsy material, a washing, a swabbing, etc., as would be well known in the art.

The detection of a polymorphism (e.g., single nucleotide polymorphism, missense, insertion/deletion (indel), substitution, insertion), genetic marker or allele of this invention can be carried out according to various protocols standard in the art and as described herein for analyzing nucleic acid samples and nucleotide sequences, as well as identifying specific nucleotides in a nucleotide sequence.

For example, nucleic acid may be obtained from any suitable sample from the subject that will contain nucleic acid and the nucleic acid can then be prepared and analyzed according to well-established protocols for the presence of genetic markers according to the methods of this invention. In some embodiments, analysis of the nucleic acid can be carried by amplification of the region of interest according to amplification protocols well known in the art (e.g., polymerase chain reaction, ligase chain reaction, strand displacement amplification, transcription-based amplification, self-sustained sequence replication (3SR), Qβ replicase protocols, nucleic acid sequence-based amplification (NASBA), repair chain reaction (RCR) and boomerang DNA amplification (BDA), etc.). The amplification product can then be sequenced (e.g., PCR based sequencing) or visualized directly in a gel by staining or the product can be detected by hybridization with a detectable probe. When amplification conditions allow for amplification of all allelic types of a genetic marker, the types can be distinguished by a variety of well-known methods, such as hybridization with an allele-specific probe, secondary amplification with allele-specific primers, by restriction endonuclease digestion, DNA or RNA sequencing and/or by electrophoresis. Thus, the present invention further provides oligonucleotides for use as primers and/or probes for detecting and/or identifying genetic markers according to the methods of this invention.

In some embodiments of this invention, detection of an allele or combination of alleles of this invention can be carried out by an amplification reaction and single base extension. In particular embodiments, the product of the amplification reaction and single base extension is spotted on a silicone chip.

In some embodiments, detection of an allele or combination of alleles of this invention can be carried out by matrix-assisted laser desorption/ionization-time of flight mass spectrometry (MALDI-TOF-MS), and sequencing methods, including, but not limited to Sanger sequencing and high-throughput sequencing methods (e.g., single-molecule real-time sequencing, ion torrent sequencing, pyrosequencing, sequencing by ligation; sequencing by synthesis; chain termination sequencing; nanopore sequencing, and the like).

Familial dilated cardiomyopathy (DCM) is a primary myocardial disease that can result in the development of congestive heart failure and sudden cardiac death. The outward symptoms of DCM include, but are not limited to, coughing, difficulty breathing/shortness of breath, episodes of collapse, exercise intolerance and/or loss of appetite. Often these symptoms are observed by owners/caretakers of these dogs prior to any diagnosis of the disease. However, occult disease can be present in a dog prior to the development of these outward symptoms. In some cases, dogs can remain occult even with advanced heart dysfunction. Until symptoms develop (e.g., coughing, difficulty breathing/shortness of breath, episodes of collapse, exercise intolerance and/or loss of appetite), the disease is considered “occult.” The term “occult” refers to a dog or disease in a dog that does not exhibit outward symptoms of the disease (asymptomatic) but exhibits at least one clinical symptom that can be diagnosed or observed by a clinician using, for example, a thoracic radiograph, a Holter monitor and/or an echocardiogram. The symptoms associated with “occult” disease in DCM include, but are not limited to, ventricular arrhythmia, supraventricular arrhythmia, decrease in heart function, heart enlargement, identification of a heart murmur, and/or elevation in brain natriuretic peptide or troponin levels. In some embodiments, occult disease includes a left ventricular end diastolic dimension of at least 4.8 cm and a fractional shortening less than about 20% (e.g., about 20, 19, 18, 17, 16, 15, 14, 13, 12, 10% and the like), without evidence of ongoing systemic disease or other congenital or acquired heart disease and/or the presence of more than about 25 ventricular premature complexes (e.g., about 25, 24, 23, 22, 21, 20, 19, 18, 17, 16, 15, 14, 13, 12, 10, 9, 8, 7, 6, 5, and the like) in a 24 hour period without other underlying causes for the arrhythmia.

A dog identified as having a mutation in the titin gene or a mutation in the titin gene and in the PDK4 gene but does not exhibit symptoms (e.g., no coughing, difficulty breathing/shortness of breath, episodes of collapse, exercise intolerance and/or loss of appetite; e.g., “outward symptoms” that may be identifiable by observation only) and shows no evidence of disease (e.g., no clinical symptoms, e.g., no heart enlargement, no arrhythmia and the like) is considered “mutation positive pre-clinical.” The term “pre-clinical” as used herein means prior to disease development and prior to showing symptoms or evidence of occult disease. “Pre-clinical” does not include occult disease.

Surprisingly, the present inventor has found that dogs identified as having the missense mutation in the titin gene (DCM2), and especially dogs identified as having both the missense mutation in the titin gene (DCM2) and the deletion mutation in the PDK4 gene (DCM1), may benefit from early intervention (e.g., monitoring and/or treatment) prior to disease development (e.g., mutation positive preclinical disease). A dog identified early and started on treatment as described herein prior to developing the disease may develop the disease later than a control dog with the same degree of penetrance but not having received the same treatment. In these cases, disease development and progression may be delayed, thereby prolonging the life of the dog. Thus, in some embodiments, the present invention is directed to identifying those dogs that may benefit from closer monitoring of occult disease to delay the development of the disease and/or increase longevity.

The terms “delaying the development of symptoms of DCM”, “delaying the onset and/or progression of DCM” in a subject that is diagnosed and treated as described herein is in comparison to a “control” subject that is an adult dog having the mutation in the titin gene (DCM2) or having the mutation in the titin gene (DCM2) and the mutation in the PDK4 gene (DCM1), but which has not been and/or is not being treated for DCM as described herein.

“Delaying symptom development” as used herein means delaying the development of symptoms including, but not limited to, cough, shortness of breath, episodes of collapse, exercise intolerance and/or loss of appetite. In some embodiments, these symptoms may be indicative of advanced or late stage disease.

“Delaying the onset and/or progression of disease” or “delaying disease development” as used herein means delaying symptom development and/or slowing of occult disease development including, but not limited to, enlargement of the heart, supraventricular arrhythmia, ventricular arrhythmia, decrease in heart function identification of a heart murmur, and/or elevation in brain natriuretic peptide or troponin levels. In some embodiments, early stage symptoms may be symptoms of occult disease.

It has been previously determined that DCM in Doberman pinscher dogs is inherited as an autosomal dominant trait, at least in some families of dogs.¹³ A splice site mutation has been identified in the PDK4 gene, encoding a mitochondrial protein that is strongly associated with the development of DCM in some families.¹⁴ However, the PDK4 mutation does not explain all cases of this familial canine disease even within this one breed, suggesting that even within a single breed of dog genetic heterogeneity is likely.¹⁵ In this study, we evaluated a family of Doberman pinschers with DCM but without the PDK4 mutation. A deleterious mutation in the titin (TTN) gene was strongly associated with DCM in this family as well as in other affected dogs. The mutation results in an amino acid change, which likely changes the structure of the protein, and is demonstrated to decrease active tension in myofibers from affected dogs. The newly identified mutation is a single nucleotide polymorphism (SNP) (C→T missense mutation) in the immunoglobulin-like domain in the I-band spanning region of TN.

Thus, in some embodiments, the present invention provides a method of identifying a Doberman pinscher dog as having familial dilated cardiomyopathy (DCM) or having an increased risk of developing familial DCM, comprising detecting in nucleic acid from the Doberman pinscher dog a missense mutation in the titin gene (TTN) (DCM2 mutation), thereby identifying the Doberman pinscher dog as having DCM or having an increased risk of developing DCM, wherein the missense mutation is a C→T mutation on canine chromosome 36:22,321,955 (e!Ensembl (ensembl.org) Gene Accession No. ENSCAFT00000022319) (CanFAM 3.1)). The site of this missense mutation may be found by searching ensemble.org (select “dog” for species and “genomic locations” for feature type; input a range around chr36:22,321,955).

In some embodiments, the present invention provides a method of identifying a Doberman pinscher dog as having familial dilated cardiomyopathy (DCM) or as having an increased risk of developing familial DCM, comprising detecting in nucleic acid of the Doberman pinscher dog (a) a missense mutation in the titin gene (TTN) (DCM2 mutation), and (b) a splice site mutation in pyruvate dehydrogenase kinase 4 (PDK4) (DCM1 mutation), wherein the missense mutation is a C→T mutation on canine chromosome 36:22,321,955 (e!Ensembl Gene Accession No. ENSCAFT00000022319) (CanFAM 3.1) and the splice site mutation in PDK4 is a 16 base pair deletion in the 5′ donor splice site of intron 10 of PDK4 on canine chromosome Chr14:20829667-20829682 (e!Ensembl Gene Accession No. ENSCAFG00000002129 (=PDK4)) (CanFAM3.1). This deletion mutation may be found by searching ensemble.org (select “dog” for species and “genomic locations” for feature type; input the site provided above of 14:20829667-20829682).

The 16 base pair sequence deletion in PDK4 (DCM1 mutation) comprises the nucleotide sequence of GTATCCTTTCAACCCA (SEQ ID NO:1). This 16-base pair deletion removes the 5′ donor site gt plus an additional 14 bases downstream to the next gt in the intron, thereby creating a presumed cryptic splice site (Meurs et al. Human Genet. 131:1319-1325 (2012)).

A subject of the present invention is a dog. In some embodiments, the subject is a Doberman pinscher dog. In some embodiments, a Doberman pinscher dog may be heterozygous for the DCM2 mutation or a Doberman pinscher dog may be homozygous for the DCM2 mutation. In some embodiments, a Doberman pinscher dog that is heterozygous or homozygous for the DCM2 mutation may also be heterozygous for the DCM1 mutation or homozygous for the DCM1 mutation.

In some embodiments, a risk of developing DCM in a Doberman pinscher dog identified as having the DCM2 mutation may be increased by about 45-55%% (e.g., about 45, 46, 47, 48, 49, 50, 51, 52, 53, 54, 55% or more) as compared to a Doberman pinscher dog that does not have a mutation in the titin gene (e.g., DCM2 mutation). In some embodiments, the risk of developing DCM in a Doberman pinscher dog identified as having the DCM2 mutation may be increased by about 50% as compared to a Doberman pinscher dog that does not have a mutation in the titin gene (e.g., DCM2 mutation). In some embodiments, a risk of developing DCM in a Doberman pinscher dog identified as having the mutation in the PDK1 gene (DCM1) and the mutation in the titin gene (DCM2) may be increased by about 60% to about 75% (e.g., about 60, 61, 62, 63, 64, 65, 66, 67, 68, 69, 70, 71, 72, 73, 74, 75% or more) as compared to a Doberman pinscher dog that does not have either of the mutation in the PDK1 gene (DCM1) and the mutation in the titin gene (DCM2). In some embodiments, a risk of developing DCM in a Doberman pinscher dog identified as having the mutation in the PDK1 gene (DCM1) and the mutation in the titin gene (DCM2) may be increased by about 65% to about 70% (e.g., about 65, 66, 67, 68, 69, 70% or more) as compared to a Doberman pinscher dog that does not have either of the mutation in the PDK1 gene (DCM1) and the mutation in the titin gene (DCM2).

In some embodiments, a method of identifying and treating a Doberman pinscher dog at increased risk for death due to familial dilated cardiomyopathy (DCM) is provided, the method comprising:

a) detecting in nucleic acid from the Doberman pinscher dog a missense mutation in the titin gene (TTN) (DCM2 mutation), thereby identifying the Doberman pinscher dog as being at increased risk of developing familial DCM and having an increased risk for death due to DCM; and b) treating the Doberman pinscher dog having an increased risk for death prior to symptom and/or disease development, thereby delaying the onset and/or progression of the disease in the Doberman pinscher dog.

In some embodiments, a method of identifying and treating a Doberman pinscher dog at increased risk for death due to familial dilated cardiomyopathy (DCM) is provided, the method comprising: a) detecting in nucleic acid of the Doberman pinscher dog (i) a missense mutation in the titin gene (TTN) (DCM2 mutation), and (ii) a splice site mutation in pyruvate dehydrogenase kinase 4 (PDK4) (DCM1 mutation), thereby identifying the Doberman pinscher dog as being at increased risk of death due to DCM; and b) treating the Doberman pinscher dog having an increased risk for death prior to symptom and/or disease development, thereby delaying the onset and/or progression of the disease in the Doberman pinscher dog.

In some embodiments, treating a Doberman pinscher dog having an increased risk for death may include, but is not limited to, administering to the dog a therapeutically effective amount of an inodilator, an angiotensin converting enzyme (ACE) inhibitor and/or a ventricular antiarrhythmic agent. These drugs may be used in alone or in any combination. Any acceptable inodilator, ACE inhibitor and/or ventricular antiarrhythmic agent known in the art for use in dogs may be used with this invention. In some embodiments, the inodilator may be pimobendan, amrinone, levosimendan, dobutamine, and/or milrinone). Pimobendan is also referred to as pimobendane and the tradenames including, but not limited to, the tradenames: Vetmedin®, Acardi®, and Heartmedin®. In some embodiments, the ACE inhibitor may be enalapril, benazepril, zofenopril, perindopril, trandolapril, captopril, lisinopril, and/or ramipril. In some embodiments, the ventricular antiarrhythmic agent may be mexiletine, sotalol, and/or amiodarone.

In some embodiments, treating may comprise supplementing the diet of a dog identified as being at increased risk of death due to DCM with free fatty acids (e.g., eicosapentaenoic acid (EPA), and/or docosahexaenoic acid (DHA).

Treating may further comprise monitoring a dog identified as being at risk for death due to DCM. In some embodiments, monitoring may comprise observing the dog for outward symptoms of the disease (e.g., coughing, difficulty breathing/shortness of breath, episodes of collapse, and the like). In some embodiments, monitoring may comprise monitoring for occult disease (e.g., ventricular arrhythmia, supraventricular arrhythmia, decrease in heart function, heart enlargement, identification of a heart murmur, and/or elevation in brain natriuretic peptide or troponin levels, and the like) using, for example, a thoracic radiograph, a Holter monitor, an echocardiogram and/or by measuring brain natriuretic peptide (bnp) and/or troponin levels, thereby detecting occult disease in advance of symptoms. A thoracic radiograph, a Holter monitor and/or an echocardiogram may be used to monitor the progression of DCM in a dog. In some embodiments, measurement of brain natriuretic peptide (bnp) and/or troponin levels may be also be used to monitor the progression of DCM in a dog. In some embodiments, monitoring an asymptomatic dog for occult disease may begin at about 2, 3, 4, 5, 6, 7, or more years of age.

Treating may further comprise directing/advising (other guidance) an owner and/or caretaker, and/or other individual involved in the care and maintenance of the dog on exercise considerations/exercise monitoring. For example, a caretaker/owner may be advised to allow the dog to set their own exercise limits. A caretaker/owner may be advised to avoid having the dog participate in regular vigorous activities such as runs exceeding, for example, one mile, or participating in competitions (e.g., agility, lure coursing, and the like). Other guidance may be to avoid exercise when the environmental temperature may result in increased stress for the dog.

Any of the methods of treating may be used alone or in any combination for treating a dog as described herein. Thus, for example, a dog at risk for developing DCM in which the DCM2 mutation is detected, or in which the DCM2 and DCM1 mutation are detected, may be treated with an inodilator and an ACE inhibitor once monitoring shows that cardiac enlargement is present, thereby delaying and/or inhibiting the progression of the disease. A dog may be asymptomatic (that is, no outward symptoms observed) at the time that treatment is initiated. A dog may be pre-clinical (no outward symptoms and no clinical disease; e.g., mutation positive pre-clinical) when treatment is initiated. In some embodiments, beginning treatment when the dog is asymptomatic or pre-clinical may slow progression of the disease and prolong the life of the dog.

In some embodiments, the present invention provides a method of treating a Doberman pinscher dog having familial dilated cardiomyopathy (DCM) or having an increased risk of developing familial DCM, comprising (a) administering to the dog a therapeutically effective amount of an inodilator, an angiotensin converting enzyme (ACE) inhibitor and/or a ventricular antiarrhythmic agent; (b) supplementing the diet of the dog with free fatty acids; (c) monitoring the dog for symptoms (observation) and/or for occult disease; and/or (d) directing/advising (other guidance) the dog's owner/caretaker on exercise considerations/exercise monitoring, wherein the Doberman pinscher dog has a missense mutation in the titin gene (TTN) (DCM2 mutation).

In some embodiments, the present invention provides a method of treating a Doberman pinscher dog having familial dilated cardiomyopathy (DCM) or having an increased risk of developing familial DCM, comprising (a) administering to the dog a therapeutically effective amount of an inodilator, an angiotensin converting enzyme (ACE) inhibitor and/or a ventricular antiarrhythmic agent; (b) supplementing the diet of the dog with free fatty acids; (c) monitoring the dog for symptoms (observation) and/or for occult disease; and/or (d) directing/advising (other guidance) the dog's caretaker on exercise considerations/exercise monitoring, wherein the Doberman pinscher dog has (a) a missense mutation in the titin gene (TTN) (DCM2 mutation) and (b) a splice site mutation in pyruvate dehydrogenase kinase 4 (PDK4) (DCM1 mutation).

In some embodiments, the present invention provides a method of delaying the onset or progression of familial dilated cardiomyopathy (DCM) or delaying the development of symptoms of DCM in a Doberman pinscher that is dog that is pre-symptomatic for familial DCM and having a missense mutation in the titin gene (TTN) (DCM2), the method comprising treating the Doberman pinscher dog by (a) administering to the dog a therapeutically effective amount of an inodilator, an angiotensin converting enzyme (ACE) inhibitor and/or a ventricular antiarrhythmic agent; (b) supplementing the diet of the dog with free fatty acids; (c) monitoring the dog for symptoms (observation) and/or for occult disease; and/or (d) directing/advising (other guidance) the dog's caretaker on exercise considerations/exercise monitoring, thereby delaying the onset and/or progression of familial dilated cardiomyopathy (DCM) or delaying the development of symptoms of DCM in the Doberman pinscher dog as compared to a Doberman pinscher dog that has the mutation in the titin gene (DCM2) but which has not been and/or is not being treated for DCM as described herein.

In some embodiments, the present invention provides a method of delaying the onset and/or progression of familial dilated cardiomyopathy (DCM) or delaying the development of symptoms of DCM in a Doberman pinscher dog that is pre-symptomatic for familial DCM and having (a) a missense mutation in the titin gene (TTN) (DCM2 mutation) and (b) a splice site mutation in pyruvate dehydrogenase kinase 4 (PDK4 (DCM1 mutation), the method comprising treating the Doberman pinscher dog by comprising treating the Doberman pinscher dog by (a) administering to the dog a therapeutically effective amount of an inodilator, an angiotensin converting enzyme (ACE) inhibitor and/or a ventricular antiarrhythmic agent; (b) supplementing the diet of the dog with free fatty acids; (c) monitoring the dog for symptoms (observation) and/or for occult disease; and/or (d) directing/advising (other guidance) the caretaker of the dog on exercise considerations/exercise monitoring, thereby delaying the onset and/or progression of familial dilated cardiomyopathy (DCM) or delaying the development of symptoms of DCM in the Doberman pinscher dog as compared to a Doberman pinscher dog that has a mutation in the titin gene (DCM2) and the DCM1 mutation but which has not been and/or is not being treated for DCM as described herein.

In some embodiments, if the risk of developing DCM is recognized early, e.g., when a dog is asymptomatic/preclinical and treatment is begun, the median time to onset of symptoms (e.g., chronic heart failure (e.g., persistent cough, breathing difficulty but semi responsive to medication)) or sudden death may be delayed for about 9 months to about 15 months (e.g., about 9, 10, 11, 12, 13, 14, 15 months) as compared to a dog having the mutation in the titin gene (DCM2) or a dog having the mutation in the titin gene (DCM2) and a mutation in the PDK4 gene (DCM1), which is not monitored and treated as described herein. In addition, the median survival time for a dog that is identified as being at risk of developing DCM, and which is monitored and treated may be increase by about 8 months to about 15 months (e.g., about 8, 9, 10, 11, 12, 13, 14, 15 months) as compared to a Doberman pinscher dog that has a mutation in the titin gene (DCM2) or has a mutation in both the titin gene (DCM2) and the mutation in the PDK4 gene (DCM1) but which has not been and/or is not being treated for DCM as described herein.

By identifying those dogs carrying the DCM2 mutation or the DCM2 and the DCM1 mutation, guidance can be given to reduce the incidence of familial dilated cardiomyopathy (DCM) in Doberman pinscher dog population. Thus, in some embodiments, a method of selecting a Doberman pinscher dog for breeding is provided, comprising selecting a Doberman pinscher dog that does not have a missense mutation in the titin gene (TTN) (DCM2). In some embodiments, a method of selecting a Doberman pinscher dog for breeding is provided, comprising selecting a Doberman pinscher dog that does not have a missense mutation in the titin gene (TTN) (DCM2) and does not have a splice site mutation in pyruvate dehydrogenase kinase 4 (PDK4) (DCM1).

In some embodiments, the present invention provides a method of selectively breeding Doberman pinscher dogs to decrease the frequency of familial dilated cardiomyopathy (DCM) in a Doberman pinscher dog breeding population, the method comprising: (a) identifying Doberman pinscher dogs in the breeding population that do not have a missense mutation in the titin gene (TTN) (DCM2); and (b) breeding those Doberman pinscher dogs that do not have a missense mutation in the titin gene (TTN) (DCM2), thereby decreasing the frequency of familial dilated cardiomyopathy (DCM) in the Doberman pinscher dog population.

In some embodiments, the present invention provides a method of selectively breeding Doberman pinscher dogs to decrease the frequency of familial dilated cardiomyopathy (DCM) in a Doberman pinscher dog breeding population, the method comprising: (a) identifying Doberman pinscher dogs in the breeding population that do not have a missense mutation in the titin gene (TTN) (DCM2) and do not have a splice site mutation in pyruvate dehydrogenase kinase 4 (PDK4) (DCM1); and (b) breeding those Doberman pinscher dogs that do not have a missense mutation in the titin gene (TTN) (DCM2) and do not have a splice site mutation in pyruvate dehydrogenase kinase 4 (PDK4) (DCM1), thereby decreasing the frequency of familial dilated cardiomyopathy (DCM) in the Doberman pinscher dog population.

The DCM2 mutation comprises a missense C→T mutation on canine chromosome 36:22,321,955 (ENSCAFT00000022319 (CanFAM 3.1)). The DCM1 mutation is a splice site mutation in PDK4, which is a 16 base pair deletion in the 5′ donor splice site of intron 10 of PDK4 on chromosome Chr14:20829667-20829682 (ENSCAFG00000002129 (=PDK4))) (CanFAM3.1)).

In some embodiments, the Doberman pinscher dog may be heterozygous for DCM1. In some embodiments, the Doberman pinscher dog may be heterozygous for DCM2. In some embodiments, the Doberman pinscher dog may be homozygous for DCM1. In some embodiments, the Doberman pinscher dog may be homozygous for DCM2.

Guidance to breeders includes, for example, ideally a dog that is positive for both DCM2 and DCM1 is not bred. However, a dog that is positive for both DCM2 and DCM1 but does not show signs of disease may be bred to a dog that is negative for both mutations. A dog that is positive for DCM2 but negative for DCM1 should not be bred to a dog that is positive for DCM1 because the combination of mutations results in a more severe expression of the disease.

The present invention further comprises a kit or kits to carry out the methods of this invention. A kit of this invention can comprise reagents, buffers, and apparatus for mixing, measuring, sorting, labeling, etc., as well as instructions and the like as would be appropriate for genotyping the DCM2 missense mutation and the DCM1 deletion in a nucleic acid sample. The kit may further comprise control reagents, e.g., to identify markers for a specific ethnicity or gender.

The invention will now be described with reference to the following examples. It should be appreciated that these examples are not intended to limit the scope of the claims to the invention, but are rather intended to be exemplary of certain embodiments. Any variations in the exemplified methods that occur to the skilled artisan are intended to fall within the scope of the invention.

### EXAMPLES

### Example 1

Dilated cardiomyopathy (DCM) is one of the most common forms of familial cardiomyopathy. The Doberman pinscher dog is a natural animal model of familial DCM used to study different aspects of the disease from pathophysiology to stem cell therapy. We have previously demonstrated that a form of canine DCM in this breed is inherited as an autosomal dominant trait, and is associated with a splice site mutation in the pyruvate dehydrogenase kinase 4 (PDK4) gene, however not all affected dogs have the PDK4 mutation. In this study, whole genome sequencing of a family of affected Doberman pinchers without the PDK4 mutation was used to identify a second genetic variant for DCM. Here we describe a pathologic variant in the titin gene that causes a missense mutation in an immunoglobulin-like domain in the I-band spanning region of the titin molecule. Protein analysis revealed normal levels of titin and unaltered isoform expression ratios. titin-based passive tension is normal in dogs with the missense mutation but active tension is reduced.

Materials and Methods:

This study was conducted in accordance with the guidelines of the North Carolina State University Institutional Animal Care and Use Committee.

Two sibling (male, female) Doberman pinchers were presented for evaluation of clinical signs consistent with congestive heart failure with DCM at the North Carolina State University College of Veterinary Medicine at 4 years of age. Their mother had died unexpectedly of sudden cardiac death at 6 years of age. The cardiac status of the father was unknown and he was not available for evaluation. Physical examination, echocardiographic and electrocardiographic examination were performed by a board-certified veterinary cardiologist. The echocardiogram was performed using standard 2 dimensional and M-mode echocardiographic methods.³⁴ Dogs were considered to be affected if they had a left ventricular end diastolic dimension of at least 4.8 cm and a fractional shortening less than 20%, without evidence of ongoing systemic disease or other congenital or acquired heart disease.³⁵

Both sibling dogs were determined to be affected. The female had been bred once and had 7 existing offspring. Six offspring were examined by a board-certified veterinary cardiologist, and five were diagnosed with DCM according to the criteria above by five years of age.

DNA samples were obtained from the two proband siblings and the six available offspring of the female proband. All affected dogs in this family lacked the previously reported PDK4 mutation. Samples from the female proband, one of her male affected offspring, and three additional (2 male, 1 female) unrelated affected Doberman pinschers lacking the PDK4 mutation were selected for whole genome sequencing.

Approximately 3 μg of DNA from each dog was submitted for library preparation and whole genome sequencing at the University of North Carolina Chapel Hill High-Throughout Sequencing Facility (https://www.med.unc.edu/genomics). All sequencing experiments were designed as 125 bp paired-end reads and samples were run on either 1 or 2 lanes of an Illumina HiSeq 2500 high-throughput sequencing system.

Variant calling from WGS data was performed using a standardized bioinformatics pipeline for all samples as described previously.³⁶ Briefly, sequence reads were trimmed using Trimmomatic 0.32 to a minimum phred-scaled base quality score of 30 at the start and end of each read with a minimum read length of 70 bp, and aligned to the canFam3 reference sequence using BWA 0.7.13.³⁷⁻³⁹ Aligned reads were prepared for analysis using Picard Tools 2.5 (http://broadinstitute.github.io/picard) and GATK 3.7 following best practices for base quality score recalibration and indel realignment (Broad Institute, Cambridge, Mass.).⁴⁰⁻⁴² Variant calls were made using GATK's HaplotyeCaller walker, and variant quality score recalibration (VQSR) was performed using sites from dbSNP 146 and the Illumina 174K CanineHD BeadChip as training resources. We applied a VQSR tranche sensitivity cutoff of 99.9% to SNPs and 99% to indels for use in downstream analyses; genotype calls with a phred-scaled quality score <20 were flagged but not removed from the variant callset.

Variants present in at least 4 of 5 affected dogs (to allow for possible areas of poor coverage) were selected and filtered against a database of variants from 125 non-Doberman pinscher dogs from 23 different dog breeds. Variants were then categorized by Variant Effect Predictor 87 and prioritized by their functional impact (e.g., stop codon, change in amino acid, etc.) as well as potential cardiac involvement (evidence of cardiac expression, previous association with some form of cardiomyopathy).⁴³ Variants of highest interest were evaluated for functional significance with Polyphen (genetics.bwh.harvard.edu/pph2), Sift (sift.jcvi.org) and Provean (provean.jcvi.org/index.php).

The most promising variants were evaluated by Sanger Sequencing of 99 affected and 122 unaffected Doberman pinchers maintained in a database at the NCSU College of Veterinary Medicine. Affected phenotype was defined as described above and the unaffected phenotype was based on the presence of echocardiographically determined normal left ventricular dimensions, a fractional shortening of at least 25% and an age of at least 10 years. The variants were tested for allelic association with DCM using a Fischer's exact test. A p-value of <0.05 was considered significant. Penetrance, relative risk and odds ratio were also determined for each of the most promising variants that were evaluated by Sanger Sequencing.

Titin isoform (N2BA and N2B) expression analysis of LV tissues was performed as previously described.^{44,45} The solubilized samples were electrophoresed on 1% Seakem Gold Agarose gels (Lonza, Allendale, N.J.) using a Vertical Agarose Gel System as previously described.⁴⁵ Gels were run for 3 hours (h) 20 minutes (min) (@15 mA per gel), stained overnight using Coomassie brilliant blue (Acros Organics, Pittsburgh, Pa.), scanned using a commercial scanner (Epson Corporation, Long Beach Calif.) and analyzed using One-D scan (Scanalytics Inc, Rockville Md.). Each sample was loaded in a range of six volumes (microliters) and the integrated optical density (OD) of titin and myosin heavy chain (MHC) were determined as a function of volume. Further, slope of the linear relationship was obtained for each protein to quantify expression ratios. For titin western blots, solubilized samples were run on a 0.8% Seakem Gold Agarose gel as described. On day 1, gels were run for 2 h 40 min, transferred onto a PVDF membrane (Immobilon-FL®, Millipore, Burlington, Mass.) using a semi-dry transfer unit (Trans-Blot Cell, Bio-Rad, Hercules Calif.) for 2 h 30 min, stained using Ponceau-S(Sigma, Burlington, Mass.) to visualize the total protein transferred and let dry overnight. On day 2, membranes were scanned and probed overnight with anti-titin N-terminus (Z1Z2, Abnova, Cat. No. H00007273-M06) and anti-titin C-terminus (M8M9, myomedix.com) antibodies. On day 3, membranes were labeled with secondary antibodies conjugated to fluorescent dyes with infrared (IR) excitation spectra (CF680, Goat anti-Rabbit, 20067-1 and CF790, Goat anti-Mouse 20342, Biotium Hayward, Calif.). Blots were then scanned using Odyssey Infrared Imaging System (LI-COR Biosciences, Lincoln, Nebr.) and the images analyzed using LI-COR software (Image Studio Lite 5.2).

Frozen LV cardiac tissues from two Doberman pinchers homozygous for the TT and from two unaffected dogs used as controls, were warmed up from −80° C. to −20° C. in a 50% glycerol/relaxing solution ((in mM): 40 BES, 10 EGTA, 6.56 MgCl2, 5.88 Na-ATP, 1 dithiothreitol (DTT), 46.35 K-propionate, 15 creatine phosphate, pH 7.0) containing protease inhibitors ((in mM): 0.1 E64, 0.47 leupeptin and 0.25 phenylmethylsulfonyl fluoride (PMSF)) for 24 h. The samples were then placed on ice and fiber bundles were dissected and then skinned in relaxing solution containing 1% Triton X-100 and protease inhibitors for 3 h at 4° C. in a rotator. Skinned fiber bundles were washed thoroughly with relaxing solution, stretched from the slack length at ˜15% in a sylgard coated petri dish and processed by conventional TEM. Briefly, fiber bundles were fixed in a mix solution of 3.7% paraformaldehyde, 3% glutaraldehyde and 0.2% tannic acid in PBS (10 mM, pH 7.2), and post fixed in 1% OsO4 in PBS for 30 minutes (min) at 4° C. Subsequently, samples were dehydrated in an ethanol graded series, infiltrated with propylene oxide, and transferred to a mix of 1:1 propylene oxide:Araldite 502/Embed 812 (Epon-812, EMS), then to a pure Araldite 502/Embed 812 resin and polymerized for 48 h at 60° C. Longitudinal sections of 100 nm were obtained with a Reichert-Jung ultramicrotome and contrasted with 1% potassium permanganate and lead citrate. Samples were observed in a TECNAI Spirit G2 transmission electron microscope (FEI) operated at 100 kV, and images acquired with a side mounted AMT Image Capture Engine V6.02 (4 Mpix) digital camera.

Mid-myocardial fibers from tissue stored at −20° C. in 50% glycerol/relaxing solution were identified and dissected out in 100-2001 μm diameter strips, approximately 1500 μm long⁴⁶. The strips were briefly washed with relaxing solution then skinned for 1 hour at 4° C. on a 2D rocker. The strips were washed with relaxing solution then aluminum T-clips attached to the ends for mounting. One end was attached to a force transducer (AE801; SensorOne) and the other to a servo-motor (308B; Aurora) in a bath of relaxing solution⁴⁷. Sarcomere Length (SL) was measured using a laser diffraction system. Active force was measured by stretching the preparation to SL 2.0 μm and activating it using a pCa 4.0 solution (in mM: 40 BES, 10 CaCO3 EGTA, 6.29 MgCl2, 6.12 Na-ATP, 1 DTT, 45.3 potassium-proprionate, 15 creatine phosphate) with protease inhibitors. Tissue producing zero active force was immediately discarded. Slack length and cross-sectional area (CSA) of the preparation was measured in order to calibrate percent stretch and normalize measured forces (tension=mg/mm²). To measure total passive tension, a stretch-hold-release protocol was used in which the tissue was stretched to a given SL within the physiological range (2.0 to 2.3 μm) at 10%/s, held for 90 s, and released back to slack length. When SL could not be reliably measured, it was assumed to track with muscle length as it does in other samples. In cases like this, the stretch calibration from an average control sample was applied. The preparation was then extracted using sequential treatments (40 min) with 0.6 M KCl in relaxing solution followed by 1.0 M KI in relaxing solution to depolymerize the thick filaments and remove the anchoring points of titin, keeping ECM intact. The stretch-hold-release protocol was then repeated to measure the ECM-based passive tension. titin-based passive tension was calculated by subtracting the ECM-based passive tension from the total passive tension.

A protein sequence analysis was used to access information on the predicted consequences of the amino acid change.^{43,48-50} The structural model was produced using the JSmol applet (www.jmol.org) embedded in the TITINdb webserver (fraternalilab.kcl.ac.uk/TITINdb). The protein sequence alignment is a subset of the titin orthologue multiple sequence alignment downloaded from Ensembl.⁵¹

Results.

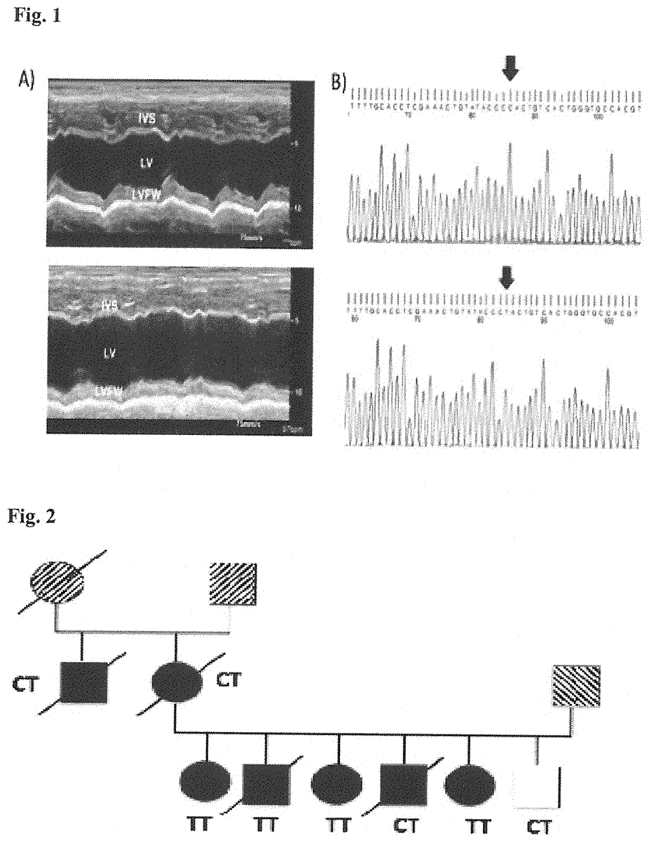

A missense C/T mutation in the titin gene at canine chromosome 36:22,321.955 (e!Ensembl (ensembl.org) Gene Accession No. ENSCAFT00000022319) (CanFAM 3.1)) segregated with the presence of DCM in a family of Doberman pinschers (FIG. 1 and FIG. 2). The mutation was not observed in any of 125 unaffected non-Doberman pinscher dogs of 23 breeds in our canine whole genome database. Two proband dogs were heterozygous, 4 of 6 affected offspring were homozygous and 2 were heterozygous for the mutation, consistent with an autosomal dominant mode of inheritance (FIG. 2). Evaluation by Sanger sequencing indicated five variants with a p<0.1, but only one, the missense C/T mutation in the titin gene was significantly associated (Table 1). Within a population of 99 affected and 122 unaffected Doberman pinschers, the titin mutation was significantly associated with disease (p=0.014). The mutation had a penetrance of 55%, a relative risk of 3.3 and an odds ratio of 6.2.

TABLE 1
Variants associated with canine DCM with a p < 0.1

| Chromosomal | | Reference | Variant | p |
|---|---|---|---|---|
| location | Gene | Allele | Allele | value |
| 1:95279812 | ROR2 | G | A | 0.07 |
| 2:73235007 | NROB2 | G | A | 0.08 |
| 17:52330510 | DENND2C | C | T | 0.08 |
| 29:27413758 | MRPS28 | C | A | 0.09 |
| 36:22321955 | TTN | C | T | <0.0001 |

The mutation is predicted to change the amino acid at this location from glycine to arginine; this glycine is highly conserved in a wide range of species and corresponds in humans to residue p.8898G>R in the immunoglobulin-like domain 171. 171 is located in the middle Tandem Ig segment of titin's molecular spring region of the cardiac N2BA isoform. The mutation is predicted to be a deleterious change by mutation prediction algorithms. Polyphen (genetics.bwh.harvard.edu/pph2), predicts the mutation to be likely damaging (score of 0.999); SIFT (sift.jcvi.org) predicts it to be a deleterious change (score of 0.03) and Provean (provean.jcvi.org/index.php) predicts it to be a deleterious change (score of −5.113).

A titin protein expression analysis was performed using left ventricular (LV) wall tissue that had been flash frozen. No major differences were found in titin expression when comparing control animals with animals that were either heterozygous for the mutation (CT) or homozygous (TT)). The total titin—myosin heavy chain ratio was unaltered (0.20±0.04 (CC), 0.17±0.03 (CT), 0.29±0.06 (TT)). A Western blot analysis using the Z1Z2 antibody that detects titin's N-terminus and the M8M9 antibody that detect titin's C-terminus revealed that both N2BA and N2B titin in mutant animals were full-length titin. There was no apparent difference in the expression ratio of N2BA:N2B titin. The ratio of T2 (large degradation product), to T1 (full-length titin) revealed a trend towards being higher in animals carrying the mutation, suggesting that there might be more protein degradation in animals carrying the mutation. However, analyzing results with an ANOVA did not provide a significant effect.

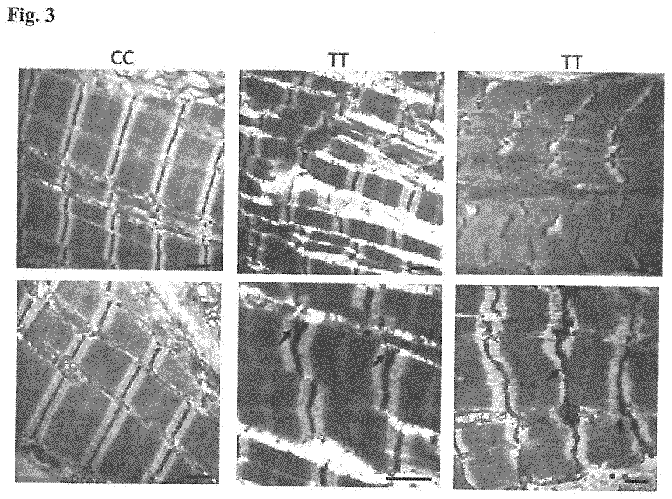

We also performed an ultrastructural and mechanical analysis on LV tissue of two control (CC) and two homozygous (TT) animals. Electron microscopy revealed focal and myopathic abnormalities in homozygous animals that include myofibrillar disarray and less dense packing of myofibrils, with sarcomeres that vary greatly in sarcomere length. In addition, Z-disk streaming is regularly observed and the Z-disks vary in thickness (FIG. 3). Similar changes including Z-disc streaming and myofibrillar disarray were observed in sections of the biceps femoris muscle of homozygous dog (IT), results not shown.

Mechanical studies were carried out on LV wall muscle strips isolated from two control (CC) and two homozygous (TT) animals. The muscle strips were maximally activated with exogenous calcium (pCa 4.0) and following their relaxation they were passively stretched to measure passive tension. Results indicate a significant reduction in maximal active tension in the homozygous animals but no difference in passive tension.

A further 46 DCM affected dogs were examined, wherein 37/46 were found to have the titin variant. Five dogs did not have either mutation, four dogs only had PDK4 (2 heterozygous, 2 homozygous), while 27 dogs had only the titin mutations (17 heterozygous, 10 homozygous). Ten dogs had both PDK4 and titin variants. These results further indicate that the titin variant is the more common variant to cause disease in canines.

Discussion.

The canine mutation reported here is in TTN, the gene most commonly associated with DCM in humans.^{2-4,16,17} Titin is the largest known protein and is expressed in both cardiac and skeletal muscle where it acts as a molecular spring contributing to both passive stiffness of muscle and active contraction, as well as biomechanical sensing and signaling.^{2,4,16,18,19} In the myocyte, titin filaments span one half of each sarcomere by anchoring the amino-terminus to the Z-disk, and the carboxy-terminus to the M-line.^{18,20,21} Titin's functional regions are named by their locations within the sarcomere including the Z-disk, I-band, A-band, and M-band regions.^{22,23} Titin gene mutations that cause DCM in people have been located throughout the molecule, consistent with the allelic heterogeneity of this disease, although most have been found in the A band region.^{4,17,21,22,24-27}

The missense mutation reported in this canine family was identified within an immunoglobulin-like domain, in humans referred to as 171²⁸. 171 is part of the middle tandem Ig segment that compromises one of the 3 spring elements within cardiac titin. 171 is included in both skeletal muscle titin and in the N2BA isoform of cardiac titin but not in the N2B cardiac isoform^{4,27-29}. Thus, the effect of the mutation is likely to be isoform specific. Although a human mutation causing DCM has not been identified in this exact location, a missense mutation has been observed within the orthologous exon in humans with an arrhythmogenic right ventricular cardiomyopathy phenotype.³⁰

It is unclear exactly how the variant identified here leads to the development of DCM. In silico programs predicted that the protein is not truncated but that rather its structure is altered. Thus it is possible that 171 is either permanently unfolded or easily unfolds when force is exerted on the domain during diastole when sarcomeres are stretched. An unfolded domain is likely to have a heightened sensitivity to degradation³¹, a notion that is supported by the increased T2 (degradation product) to T1 (full length molecule) ratio. Such proteolysis would be expected to reduce passive tension, but the effect is likely to be modest because there is less N2BA titin (171 is included) than N2B titin (171 is absent). Indeed there was no difference in the measured titin-based passive tension. This is in contrast to a recent study by Vikhorev et al³² on human cardiac myofibrils from patients with DCM due to truncation mutations in TTN (TTNtv); the authors speculated that the reduced passive tension is important in the pathology of TTNtv DCM. Our study reveals that DCM can develop with passive tension is normal.

The active tension that we obtained also contrasts with the study of Vikhorev et al³² in that we found that maximal active tension is reduced whereas Vikhorev et al³² found no difference. The reduced active tension of our study is consistent with that of myofibers engineered from human iPSC-CMs heterozygous for TTNtv, which also showed a deficit in active tension generation.³³ It is not clear how a missense mutation in 171 directly leads to a reduced level of maximal active tension and it seems likely therefore that the active tension reduction is a secondary effect that arises from the myofibrillar disarray and less dense packing of myofibrils observed in our electron microscopy study. Less dense packing of myofibrils and sarcomere disorganization is also observed in engineered heart tissues consisting of iPSC derived cardiac myocytes obtained from patients with TTNtv³³. Without being bound by any particular scientific theory, the missense mutation in 171 may lead to less dense packing of myofibrils and the ensuing overall tension reduction at the level of the myocyte may result in pathological remodeling and DCM.

The identification of a mutation in the titin gene in this spontaneous canine model of DCM completes the characterization of this large animal model of familial DCM. As such, it is an excellent model to improve our understanding of genotypic phenotypic relationships, penetrance and expression of disease and the pathophysiology of an I-band variant in the titin gene.

### REFERENCES

1 Morales, A. & Hershberger, R. E. The Rationale and Timing of Molecular Genetic Testing for Dilated Cardiomyopathy. Can J Cardiol 31, 1309-1312, doi:10.1016/j.cjca.2015.06.034 (2015).
2 de Gonzalo-Calvo, D. et al. Familial dilated cardiomyopathy: A multidisciplinary entity, from basic screening to novel circulating biomarkers. Int J Cardiol 228, 870-880, doi:10.1016/j.ijcard.2016.11.045 (2017).
3 Franaszczyk, M. et al. titin Truncating Variants in Dilated Cardiomyopathy—Prevalence and Genotype-Phenotype Correlations. PLoS One 12, e0169007, doi:10.1371/journal.pone.0169007 (2017).
4 Herman. D. S. et al. Truncations of titin causing dilated cardiomyopathy. N Engl J Med 366, 619-628, doi:10.1056/NEJMoa1110186 (2012).
5 Hershberger, R. E., Hedges, D. J. & Morales, A. Dilated cardiomyopathy: the complexity of a diverse genetic architecture. Nat Rev Cardiol 10, 531-547, doi:10.1038/nrcardio.2013.105 (2013).
6 Towbin, J. A. Inherited cardiomyopathies. Circ J 78, 2347-2356 (2014).
7 Cheng, Y., Hogarth, K. A., O'Sullivan, M. L., Regnier, M. & Pyle, W. G. 2-Deoxyadenosine triphosphate restores the contractile function of cardiac myofibril from adult dogs with naturally occurring dilated cardiomyopathy. Am J Physiol Heart Circ Physiol 310, H80-91, doi:10.1152/ajpheart.00530.2015 (2016).
8 Hensley, M. T. et al. Intracoronary allogeneic cardiosphere-derived stem cells are safe for use in dogs with dilated cardiomyopathy. J Cell Mol Med 21, 1503-1512, doi:10.1111/jcmm.13077 (2017).
9 Martin, M. W., Stafford Johnson, M. J., Strehlau, G. & King, J. N. Canine dilated cardiomyopathy: a retrospective study of prognostic findings in 367 clinical cases. J Small Anim Pract 51, 428-436, doi:10.111/j.1748-5827.2010.00966.x (2010).
10 Calvert, C. A., Pickus, C. W., Jacobs, G. J. & Brown, J. Signalment, survival, and prognostic factors in Doberman pinschers with end-stage cardiomyopathy. J Vet Intern Med 11, 323-326 (1997).
11 Petric, A. D., Stabej, P. & Zemva, A. Dilated cardiomyopathy in Doberman Pinschers: Survival, Causes of Death and a Pedigree Review in a Related Line. J Vet Cardiol 4, 17-24, doi:10.1016/S 1760-2734(06)70019-4 (2002).
12 Mausberg, T. B. et al. A locus on chromosome 5 is associated with dilated cardiomyopathy in Doberman Pinschers. PLoS One 6, e20042, doi:10.1371/journal.pone.0020042 (2011).
13 Meurs, K. M. et al. A prospective genetic evaluation of familial dilated cardiomyopathy in the Doberman pinscher. J Vet Intern Med 21, 1016-1020 (2007).
14 Meurs, K. M. et al. A splice site mutation in a gene encoding for PDK4, a mitochondrial protein, is associated with the development of dilated cardiomyopathy in the Doberman pinscher. Hum Genet 131, 1319-1325, doi:10.1007/s00439-012-1158-2 (2012).
15 Owczarek-Lipska, M. et al. A 16-bp deletion in the canine PDK4 gene is not associated with dilated cardiomyopathy in a European cohort of Doberman Pinschers. Anim Genet 44, 239, doi:10.1111/j.1365-2052.2012.02396.x (2013).
16 Kimura, A. Molecular genetics and pathogenesis of cardiomyopathy. J Hum Genet 61, 41-50, doi:10.1038/jhg.2015.83 (2016).
17 LeWinter, M. M. & Granzier, H. L. Cardiac titin and heart disease. J Cardiovasc Pharmacol 63, 207-212, doi:10.1097/FJC.0000000000000007 (2014).
18 Linke, W. A. titin Gene and Protein Functions in Passive and Active Muscle. Annu Rev Physiol 80, 389-411, doi:10.1146/annurcv-physiol-021317-121234 (2018).
19 Granzier, H. L. et al. Truncation of titin's elastic PEVK region leads to cardiomyopathy with diastolic dysfunction. Circ Res 105, 557-564, doi:10.1161/CIRCRESAHA.109.200964 (2009).
20 LeWinter, M. M. & Granzier, H. Cardiac titin: a multifunctional giant. Circulation 121, 2137-2145, doi:10.1161/CIRCULATIONAHA.109.860171 (2010).
21 Neiva-Sousa, M., Almeida-Coelho, J., Falcao-Pires, I. & Leite-Moreira, A. F. titin mutations: the fall of Goliath. Heart Fail Rev 20, 579-588, doi:10.1007/s10741-015-9495-6 (2015).
22 Gigli, M. et al. A Review of the Giant Protein titin in Clinical Molecular Diagnostics of Cardiomyopathies. Front Cardiovasc Med 3, 21, doi:10.3389/fcvm.2016.00021 (2016).
23 Granzier, H. & Labeit, S. Cardiac titin: an adjustable multi-functional spring. J. Physiol 541, 335-342 (2002).
24 Gerull, B. et al. Identification of a novel frameshift mutation in the giant muscle filament titin in a large Australian family with dilated cardiomyopathy. J Mol Med (Berl) 84, 478-483, doi:10.1007/s00109-006-0060-6 (2006).
25 Gerull, B. et al. Mutations of TTN, encoding the giant muscle filament titin, cause familial dilated cardiomyopathy. Nat Genet 30, 201-204, doi:10.1038/ng815 (2002).
26 Itoh-Satoh, M. et al. titin mutations as the molecular basis for dilated cardiomyopathy. Biochem Biophys Res Commun 291, 385-393, doi:10.1006/bbrc.2002.6448 (2002).
27 Schafer, S. et al. titin-truncating variants affect heart function in disease cohorts and the general population. Nat Genet 49, 46-53, doi:10.1038/ng.3719 (2017).
28 Bang, M. L. et al. The complete gene sequence of titin, expression of an unusual approximately 700-kDa titin isoform, and its interaction with obscurin identify a novel Z-line to l-band linking system. Circ Res 89, 1065-1072 (2001).
29 Labeit, S. et al. Expression of distinct classes of titin isoforms in striated and smooth muscles by alternative splicing, and their conserved interaction with filamins. J Mol Biol 362, 664-681, doi:10.1016/j.jmb.2006.07.077 (2006).
30 Taylor, M. et al. Genetic variation in titin in arrhythmogenic right ventricular cardiomyopathy-overlap syndromes. Circulation 124, 876-885, doi:10.1161/CIRCULATIONAHA.110.005405 (2011).
31 Anderson, B. R., Bogomolovas, J., Labeit, S. & Granzier, H. Single molecule force spectroscopy on titin implicates immunoglobulin domain stability as a cardiac disease mechanism. J Biol Chem 288, 5303-5315, doi:10.1074/jbc.M112.401372 (2013).
32 Vikhorev, P. G. et al. Abnormal contractility in human heart myofibrils from patients with dilated cardiomyopathy due to mutations in TTN and contractile protein genes. Sci Rep 7, 14829, doi:10.1038/s41598-017-13675-8 (2017).
33 Hinson, J. T. et al. HEART DISEASE. titin mutations in iPS cells define sarcomere insufficiency as a cause of dilated cardiomyopathy. Science 349, 982-986, doi:10.1126/science.aaa5458 (2015).
34 Thomas, W. P. et al. Recommendations for standards in transthoracic two-dimensional echocardiography in the dog and cat. Echocardiography Committee of the Specialty of Cardiology, American College of Veterinary Internal Medicine. J Vet Intern Med 7, 247-252 (1993).
35 Meurs, K. M., Hendrix, K. P. & Norgard, M. M. Molecular evaluation of five cardiac genes in Doberman Pinschers with dilated cardiomyopathy. Am J Vet Res 69, 1050-1053, doi:10.2460/ajvr.69.8.1050 (2008).
36 Friedenberg, S. G. & Meurs, K. M. Genotype imputation in the domestic dog. Mamm Genome 27, 485-494, doi:10.1007/s00335-016-9636-9 (2016).
37 Bolger, A. M., Lohse, M. & Usadel, B. Trimmomatic: a flexible trimmer for Illumina sequence data. Bioinformatics 30, 2114-2120, doi:10.1093/bioinformatics/btu170 (2014).
38 Li, H. & Durbin, R. Fast and accurate short read alignment with Burrows-Wheeler transform. Bioinformatics 25, 1754-1760, doi:10.1093/bioinformatics/btp324 (2009).
39 Lindblad-Toh, K. et al. Genome sequence, comparative analysis and haplotype structure of the domestic dog. Nature 438, 803-819, doi:10.1038/nature04338 (2005).
40 DePristo, M. A. et al. A framework for variation discovery and genotyping using next-generation DNA sequencing data. Nat Genet 43, 491-498, doi:10.1038/ng.806 (2011).
41 McKenna, A. et al. The Genome Analysis Toolkit: a MapReduce framework for analyzing next-generation DNA sequencing data. Genome Res 20, 1297-1303, doi:10.1101/gr.107524.110 (2010).
42 Van der Auwera, G. A. et al. From FastQ data to high confidence variant calls: the Genome Analysis Toolkit best practices pipeline. Curr Protoc Bioinformatics 43, 11 10 11-33, doi:10.1002/0471250953.bi1110s43 (2013).
43 McLaren, W. et al. The Ensembl Variant Effect Predictor. Genome Biol 17, 122, doi:10.1186/s13059-016-0974-4 (2016).
44 Lahmers, S., Wu, Y., Call, D. R., Labeit, S. & Granzier, H. Developmental control of titin isoform expression and passive stiffness in fetal and neonatal myocardium. Circ Res 94, 505-513, doi:10.1161/01.RES.0000115522.52554.86 (2004).
45 Warren, C. M., Krzesinski, P. R. & Greaser, M. L. Vertical agarose gel electrophoresis and electroblotting of high-molecular-weight proteins. Electrophoresis 24, 1695-1702, doi:10.1002/elps.200305392 (2003).
46 Ottenheijm, C. A. et al. Altered myofilament function depresses force generation in patients with nebulin-based nemaline myopathy (NEM2). J Struct Biol 170, 334-343, doi:10.1016/j.jsb.2009.11.013 (2010).
47 Granzier, H. L. & Labeit, S. The giant muscle protein titin is an adjustable molecular spring. Exerc Sport Sci Rev 34, 50-53 (2006).
48 Laddach, A., Gautel, M. & Fraternali, F. TITINdb-a computational tool to assess titin's role as a disease gene. Bioinformatics 33, 3482-3485, doi:10.1093/bioinformatics/btx424 (2017).
49 Pires, D. E., Ascher, D. B. & Blundell, T. L. DUET: a server for predicting effects of mutations on protein stability using an integrated computational approach. Nucleic Acids Res 42, W314-319, doi:10.1093/nar/gku411 (2014).
50 Pires, D. E., Ascher, D. B. & Blundell, T. L. mCSM: predicting the effects of mutations in proteins using graph-based signatures. Bioinformatics 30, 335-342, doi:10.1093/bioinformatics/btt691 (2014).
51 Vilella, A. J. et al. EnsemblCompara GeneTrees: Complete, duplication-aware phylogenetic trees in vertebrates. Genome Res 19, 327-335, doi:10.1101/gr.073585.107 (2009).

The foregoing is illustrative of the present invention, and is not to be construed as limiting thereof. The invention is defined by the following claims, with equivalents of the claims to be included therein.

That which is claimed is:

1. A method of identifying a Doberman pinscher dog as having familial dilated cardiomyopathy (DCM) or having an increased risk of developing familial DCM, comprising detecting in nucleic acid from the Doberman pinscher dog a missense mutation in the titin gene (TTN) (DCM2 mutation), thereby identifying the Doberman pinscher dog as having DCM or having an increased risk of developing DCM,
wherein the missense mutation is a C→T mutation on canine chromosome 36:22,321,955 (e!Ensembl Gene Accession No. ENSCAFT00000022319) (CanFAM 3.1).

2. The method of claim 1, further comprising detecting in nucleic acid of the Doberman pinscher dog a splice site mutation in pyruvate dehydrogenase kinase 4 (PDK4),
wherein the splice site mutation in PDK4 is a 16 base pair deletion in the 5′ donor splice site of intron 10 of PDK4 [on chromosome Chr14:20829667-20829682] ((e!Ensembl Gene Accession No. ENSCAFG00000002129 (=PDK4)) (CanFAM3.1).

3. A method of identifying and treating a Doberman pinscher dog at increased risk for death due to familial dilated cardiomyopathy (DCM), comprising:
a) detecting in nucleic acid from the Doberman pinscher dog a missense mutation in the titin gene (TTN) (DCM2 mutation), thereby identifying the Doberman pinscher dog as being at increased risk of developing familial DCM and having an increased risk for death; and
b) treating the Doberman pinscher dog having an increased risk for death prior to symptom and/or disease development, thereby delaying the onset and/or progression of the disease in the Doberman pinscher dog.

4. The method of claim 3, further comprising:
detecting in nucleic acid of the Doberman pinscher dog a splice site mutation in pyruvate dehydrogenase kinase 4 (PDK4) (DCM1 mutation).

5. The method of claim 3, wherein treating comprises (a) administering to the dog a therapeutically effective amount of an inodilator, an angiotensin converting enzyme (ACE) inhibitor and/or a ventricular antiarrhythmic agent; (b) supplementing the diet of the dog with free fatty acids; (c) monitoring the dog for symptoms (observation) and/or for occult disease; and/or (d) directing/advising (other guidance) the dog's caretaker on exercise considerations/exercise monitoring.

6. The method of claim 3, wherein monitoring for occult disease comprises the use of a thoracic radiograph, a Holter monitor and/or an echocardiogram.

7. The method of claim 6, wherein symptoms comprise coughing, difficulty breathing/shortness of breath, episodes of collapse, exercise intolerance and/or loss of appetite, and occult disease comprises ventricular arrhythmia, supraventricular arrhythmia, decrease in heart function, heart enlargement, identification of a heart murmur, and/or elevation in brain natriuretic peptide or troponin levels.

8. A method of delaying the onset and/or progression or delaying the development of symptoms of familial dilated cardiomyopathy (DCM) in a Doberman pinscher dog that is pre-symptomatic for familial DCM and having a missense mutation in the titin gene (TTN) (DCM2), comprising treating the Doberman pinscher dog by (a) administering to the dog a therapeutically effective amount of an inodilator, an angiotensin converting enzyme (ACE) inhibitor and/or a ventricular antiarrhythmic agent; (b) supplementing the diet of the dog with free fatty acids; (c) monitoring the dog for symptoms (observation) and/or for occult disease; and/or (d) directing/advising (other guidance) the caretaker of the dog on exercise considerations/exercise monitoring, thereby delaying the onset and/or progression of familial dilated cardiomyopathy (DCM) or delaying the development of symptoms of DCM in the Doberman pinscher dog as compared to a Doberman pinscher dog that has a mutation in the titin gene (DCM2) but which has not been and/or is not being treated for DCM.

9. The method of claim 8, wherein the Doberman pinscher dog that is pre-symptomatic for familial DCM and having a missense mutation in the titin gene (TTN) (DCM2 mutation) further has a splice site mutation in pyruvate dehydrogenase kinase 4 (PDK4 (DCM1 mutation), thereby delaying the onset and/or progression of familial dilated cardiomyopathy (DCM) or delaying the development of symptoms of DCM in the Doberman pinscher dog as compared to a Doberman pinscher dog that has the mutation in the titin gene DCM2 and the mutation in the PDK4 gene (DCM1) but which has not been and/or is not being treated for DCM.

10. A method of selecting a Doberman pinscher dog for breeding, comprising selecting a Doberman pinscher dog that does not have a missense mutation in the titin gene (TTN) (DCM2).

11. The method of claim 10, wherein the Doberman pinscher dog that does not have a missense mutation in the titin gene (TTN) (DCM2) also does not have a splice site mutation in pyruvate dehydrogenase kinase 4 (PDK4) (DCM1).

12. The method of claim 10, further comprising:
identifying Doberman pinscher dogs in the breeding population that do not have a missense mutation in the titin gene (TTN) (DCM2).

13. The method of claim 11, further comprising:
identifying Doberman pinscher dogs in the breeding population that do not have a missense mutation in the titin gene (TTN) (DCM2) and do not have a splice site mutation in pyruvate dehydrogenase kinase 4 (PDK4) (DCM1).

14. The method of claim 3, wherein the missense mutation is a C→T mutation on canine chromosome 36:22,321,955 (ENSCAFT00000022319 (CanFAM 3.1)).

15. The method of claim 4, wherein the splice site mutation in PDK4 is a 16 base pair deletion in the 5′ donor splice site of intron 10 of PDK4 on chromosome Chr14:20829667-20829682 (ENSCAFG00000002129 (=PDK4)) (CanFAM3.1).

16. The method of claim 8, wherein the missense mutation is a C→T mutation on canine chromosome 36;22,321,955 (ENSCAFT00000022319 (CanFAM 3.1)).

17. The method of claim 9, wherein the splice site mutation in PDK4 is a 16 base pair deletion in the 5′ donor splice site of intron 10 of PDK4 on chromosome Chr14:20829667-20829682 (ENSCAFG00000002129 (=PDK4)) (CanFAM3.1).

18. The method of claim 12, wherein the missense mutation is a C→T mutation on canine chromosome 36:22,321,955 (ENSCAFT00000022319 (CanFAM 3.1)).

19. The method of claim 13, wherein the splice site mutation in PDK4 is a 16 base pair deletion in the 5′ donor splice site of intron 10 of PDK4 on chromosome Chr14:20829667-20829682 (ENSCAFG00000002129 (=PDK4)) (CanFAM3.1).


SEQUENCE LISTING (1 sequence)

SEQ ID NO: 1 - DNA, 16 bp, Canis familiaris
gtatcctttcaaccca